(19) United States
(12) Patent Application Publication
(10) Pub. No.: US 2014/0142522 A1
(43) Pub. Date: May 22, 2014

(54) MOIST DRESSING WITH HONEYCOMB PATTERN AND DRAIN CHANNELS

(71) Applicant: NU TECHNOLOGY, LLC, Union Dale, PA (US)

(72) Inventors: Irina V. Filippova, Union Dale, PA (US); Leonid K. Filippov, Union Dale, PA (US)

(73) Assignee: NU TECHNOLOGY, LLC, Uniion Dale, PA (US)

(21) Appl. No.: 13/678,613

(22) Filed: Nov. 16, 2012

(51) Int. Cl. A61M 35/00

(52) U.S. Cl. CPC A61M 35/00
USPC 604/290; 604/304

(57) ABSTRACT

A bandage includes a generally planar substrate having a first surface and a second surface opposed from the first surface. A first matrix extends outwardly from the first surface. The first matrix has a first plurality of reservoirs. A second matrix extends outwardly from the first surface. The second matrix has a second plurality of reservoirs. The second matrix is separated from the first matrix by a drain channel. A method of treating a wound using the bandage is also disclosed.

### FIELD OF THE INVENTION

The present invention relates to a moist dressing system that uses a honeycomb pattern for retaining treatment creams and drain channels to drain waste fluids from a burn and/or a wound site in a patient.

### BACKGROUND OF THE INVENTION

Burns and other skin wounds on not only traumatic in and of themselves, but the opening of the skin due to the burn/wound invites infection and other secondary medical issues.

In its early stage, the inflammation process in a pure burn/wound is preceded by bleedings, extravasations, and blood coagulation factors including leukocyte migration into the burn/wound from surrounding tissues and blood vessels. In a later phase (after about 24 hours), poorly developed monocytes (containing larger amounts of extracellular burn/wound material) are located in the perivascular connective tissue adjacent to the burn/wound. It is crucially important in the healing process at the burn/wound.

Prior art dressing materials used in the cleaning process of a burn/wound coincide with increase in growth factors and cytokines stimulating migration, proliferation, and differentiation of burn/wound cells providing the healing. If the burn/wound is the inflammation phase, the following three goals by must be achieved: burn/wound cleaning, debridement, and disinfection. The first goal is typically achieved with hypertonic solutions, water-soluble ointments, and proteolytic enzymes. The second goal is typically achieved with chemotherapy and antiseptics. The third goal is typically achieved by means of burn/wound draining.

It would be beneficial to provide a dressing that aids in healing of the burn/wound by retaining a cream/ointment as well as assisting in draining the burn/wound. Additionally, it would be beneficial to provide a cream/ointment that is water-soluble to assist in fluid drainage and to enhance the healing process.

### BRIEF SUMMARY OF THE INVENTION

Briefly, the present invention provides a bandage comprising a generally planar substrate having a first surface and a second surface opposed from the first surface. A first matrix extends outwardly from the first surface. The first matrix has a first plurality of reservoirs. A second matrix extends outwardly from the first surface. The second matrix has a second plurality of reservoirs. The second matrix is separated from the first matrix by a drain channel.

Additionally, the present invention provides a method of treating a wound comprising the steps of using the bandage described above; applying a cream to the pluralities of reservoirs; and applying the bandage to a patient such that the cream contacts the patient's and the drain channel extends in a non-horizontal direction respect to the patient.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a top plan view of a dressing bandage according to an exemplary embodiment of the present invention;

FIG. 2 is an enlarged view of a honeycomb shaped reservoir used in the bandage of FIG. 1;

FIG. 3 is a sectional view of the bandage of FIG. 1; and

FIG. 4 is a schematic view of the bandage of FIG. 1 applied to a patient.

### DETAILED DESCRIPTION OF THE INVENTION

In the drawings, like numerals indicate like elements throughout. Certain terminology is used herein for convenience only and is not to be taken as a limitation on the present invention. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

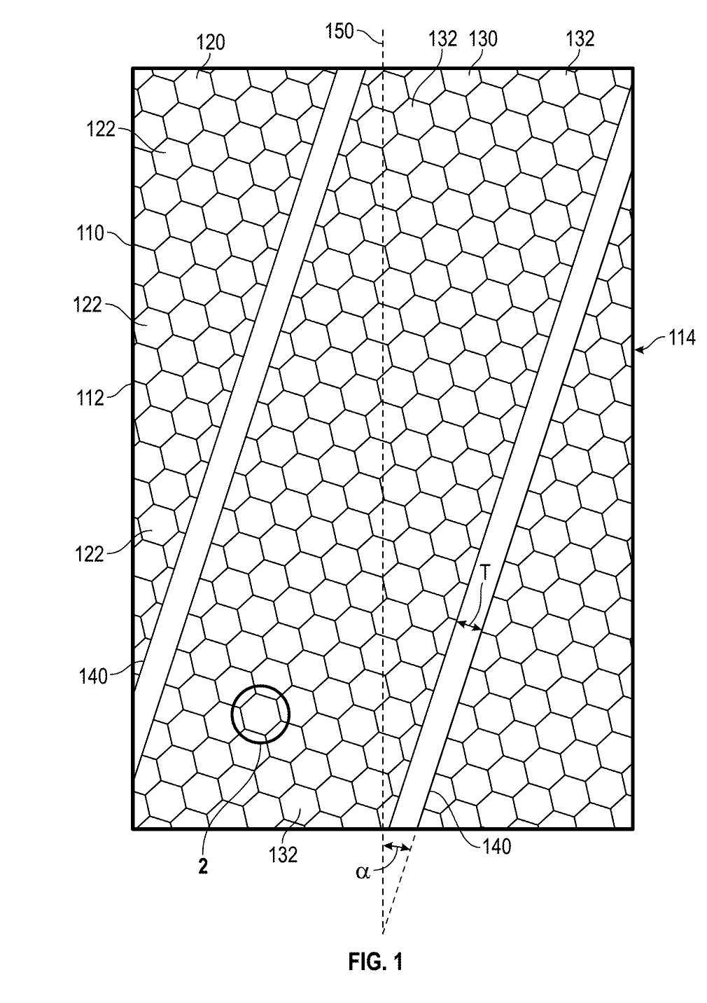

Referring to FIGS. 1-3, a dressing bandage 100 according to an exemplary embodiment of the present invention is shown. Dressing bandage 100 can be used in conjunction with a cream or ointment and applied to a burn and/or a wound in order to treat and heal the burn/wound.

Burn/wound dressing bandage 100 keeps a burn/wound area moist and bacteria-free, which is crucial to healing process. Dressing bandage 100 is applied over the burn/wound to pull fluids and exudates that drain from the burn/wound site. Testing bandage 100 keeps the burn/wound area moist and provides an anti-inflammatory, anti-bacterial, and anti-microbial environment around the burn/wound surface and allows a healthcare professional to visibly control the patient's recovery process. Compositions of inventive natural creams that are applied with dressing bandage 100 contain unsaturated natural oils and natural biological active ingredients in the form of the water-soluble complex emulsion/suspension that impart bacteria-stasis and penetrating properties.

The combination of the burn/wound dressing bandage 100 and inventive cream is a therapeutic, nontoxic, water-soluble and bio-degradable composition that provides the following advantages: (1) the dressing bandage 100 provides a protective barrier and prevents the burn/wound from progressing through healthy tissue; (2) the cream is a sterile, water-soluble composition is non-toxic and a skin non-irritant; (3) the cream moisturizes the burn/wound area, soothing the skin and thereby minimizing trauma or tissue damage; and (4) the cream is safe for use on all types of burns/wounds.

Depending upon the gravity of the lesion, a burn/wound may be of first, second or third degree. If the superficial layers of the epidermis are characterized by local erytherma (redness) and light edema (swelling), then the burn is of the first degree. Second degree burns involve a damage extended to the dermis, more marked ederma and formation of blisters containing serous exudates. Third degree burns are a true destruction of the structural elements of the skin and a formation of blisters, sores, charred zones and shock, and acute intoxication.

A principal purpose of a dressing over a burn/wound is the prevention of burn/wound desiccation. Inflammation of the burn/wound area, including neutrophil and macrophage migration, is one of the principal problems in the healing process. Dressing bandage 100 may enhance cell migration and the function of burn/wound healing as a whole. The primary contact of dressing 100 with blood surrounding the burn/wound may induce a rapid complement and coagulation factors binding, resulting in complementary leukocyte activation, chemotaxis, and cytokine production. In a moist environment of synthetic dressing neutrophils and macrophages, burn/wound cells process a high rate of cleaning and destroying, degrading, and removal of non-vital tissue. A synthetic dressing, such as dressing bandage 100, containing an extracellular matrix protein, can promote migration and proliferation of cells, contributing to burn/wound healing.

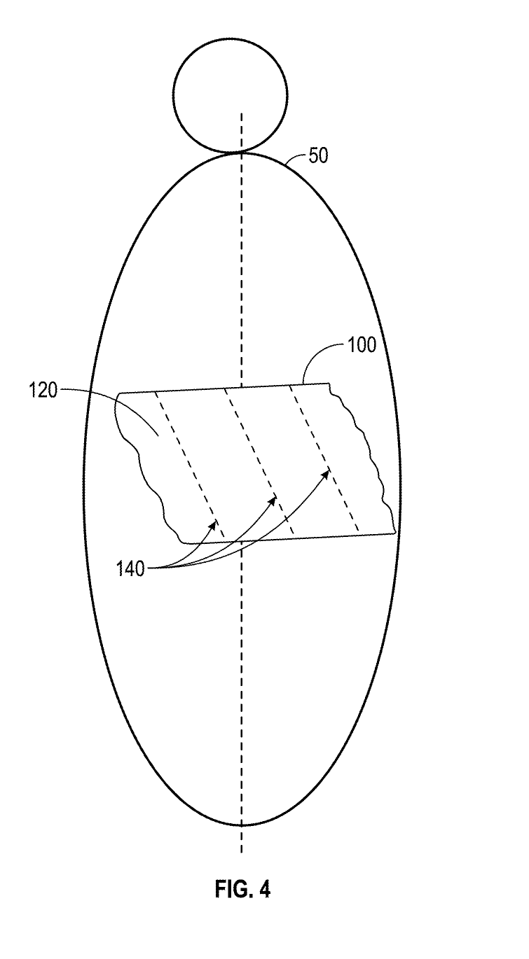

Referring to FIGS. 1-3, bandage 100 includes a generally planar substrate 110 having a first surface on 12 and a second surface opposed 114 from first surface 112. Substrate 110 can be adhesive free so that no adhesive can inadvertently contact a wound or burned skin. Bandage 100 can be completely wrapped around a patient 50 (shown in FIG. 4) and then secured to itself via tape (not shown). In an exemplary embodiment, bandage 100 comprises a translucent material and in another exemplary embodiment, bandage 100 comprises a transparent material. Bandage 100 comprises a polymer film. Exemplary polymer materials include non-adhesive polyethylene. Alternatively, bandage 100 may include a silicone material.

A first matrix 120 extends outwardly from first surface 112. First matrix 120 has a first plurality of reservoirs 122. A second matrix 130 extends outwardly from first surface 112. Second matrix 130 has a second plurality of reservoirs 132. Second matrix 130 is separated from first matrix 120 by a drain channel 140.

Substrate 110 further includes a longitudinal axis 150 and drain channel 140 is aligned such that drain channel 140 extends obliquely to longitudinal axis 150 along an angle a. In an exemplary embodiment, angle a extends at an angle between about 30° and about 60° relative to longitudinal axis 150. In an alternative exemplary embodiment, angle a extends at an angle of about 45° relative to longitudinal axis 150.

In an exemplary embodiment, a thickness T of drain channel 140 is between about 1 mm at about 5 mm and a distance between adjacent drain channels 140 is between about 5 mm and about 20 mm. Drain channel 140 is used to allow fluid to drain away from dressing 100.

Each of the plurality of reservoirs 122, 132 has a polygonal shape. In an exemplary embodiment, the shape is hexagonal and the plurality of hexagon shapes in each matrix 120, 130 forms a honeycomb pattern. The honeycomb pattern provides several advantageous properties including maximizing volume of the container of healing composition inside the honeycomb with a minimum surrounding surface as well as providing mechanical stiffness and stability under pressure.

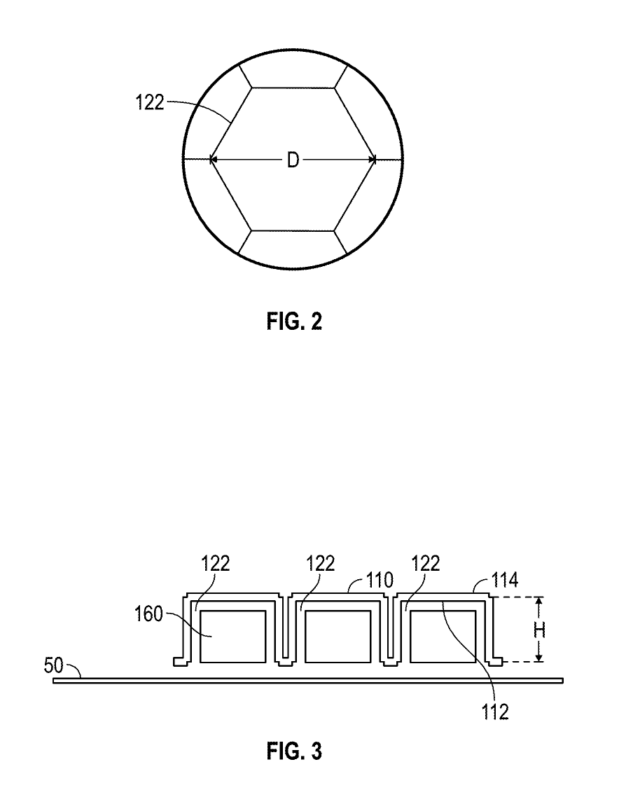

As shown FIG. 2, each reservoir 122, 232 has a maximum distance D between APCs of between about 1 mm and about 8 mm. Further, as shown FIG. 3, each reservoir 122, 232 has a height H of between about 1 mm and about 5 mm.

The honeycomb pattern of matrices 120, 130 and drain channel 150 can be formed by a hot embossing roller, as will be recognized by those having ordinary skill in the art.

In an exemplary embodiment, a fluid 160 is disposed inside each of the first and second pluralities of reservoirs 122, 232. In an exemplary embodiment, fluid 160 comprises a cream or ointment, and further, the cream may comprise a water-soluble emulsion.

Exemplary creams that may be used with dressing bandage 100 are described below. Those skilled in the art, however, will recognize that additional creams, fluids, and/or ointments not specifically discussed may be used with dressing bandage 100 within the scope of the present invention.

The inventors have formulated several compositions of cream that may be used with dressing bandage 100 using a natural ingredients base in the form of a water-soluble complex emulsion/suspension using the technology as disclosed in U.S. Pat. No. 5,393,461 entitled “PREPARATION OF STABLE AQUEOUS EMULSION OF WATER-INSOLUBLE PARTICLES”, which is incorporated herein by reference in its entirety. The inventive cream is in the form of a stable water-soluble emulsion/suspension in a temperature range between about 59° F. (about 15° C.) and about 95° F. (about 35° C.).

The microencapsulation process disclosed in U.S. Pat. No. 5,393,461 is a process that enhances through-the-skin penetration of biological active ingredients through the normal, dry, and mature skin. The complex emulsion/suspension (herein after referred to simply as an “emulsion”) disclosed in U.S. Pat. No. 5,393,461 is an emulsion in which a dispersed phase contains another dispersed phase. The multiple emulsion is a water/oil/water (W/O/W) emulsion. A W/O/W emulsion is a system in which water globules are dispersed in oil globules, the latter being themselves dispersed in an aqueous phase. Multiple emulsions are sometimes called triple-phase emulsions. Multiple emulsion includes two immiscible liquids (water or oil); therefore, their preparation demands the presence of two emulsifiers (the primary and the secondary surfactants). A solid part of the product (or cream) consists of fine particles (with size of less than about 1 micron) of sulfur and zinc oxide.

An active ingredient of the cream is microencapsulated water-soluble USP ibuprofen. Ibuprofen, in a class of drugs called nonsteroidal anti-inflammatory drug (NSAID), belonging to the group of propionic acid derivatives, inhibits the enzyme cyclo-oxygenase (prostaglandin synthesis), which catalyzes the transformation of unsaturated fatty acids to prostaglandins. It is believed that the inhibition of the prostaglandin synthesis is the cause for the analgesic, antipyreti, and anti-inflammatory action of the drug. Ibuprofen works by reducing hormones that cause inflammation and pain in the body. Ibuprofen is used to reduce the fever, pain, inflammation, and stiffness caused by many conditions.

The inventors recognize that long-term or extensive ingestion of NSAID can result in the drugs having toxicity to the kidneys and also to the lining of the stomach, possibly causing ulcers. Therefore, inventors are using the technology disclosed in U.S. Pat. No. 5,393,461 to produce microencapsulated USP ibuprofen in the form of a stable water-soluble emulsion for burn/wound/injury/scar treatment.

Exemplary compositions of a cream for burn/wound/injury/scar treatment contain water-soluble microencapsulated ibuprofen and natural biological active ingredient, including sulfur, zinc oxide, vitamin C and vitamin A. The inventors have found that the inventive creams reduce inflammation and pain, stimulate antibacterial and antimicrobial activities, decrease transdermal water loss, reduce irritation, itching, and discomfort of the skin, and stimulate of blood circulation.

The inventive creams using water-soluble microencapsulated ibuprofen and/or other NSAIDs (between about 0% and about 15% by weight) include at least some of the following:

(i) Arylpropionic acids (e.g., ibuprofen, flurbiprofen, fenoprofen, naproxen, and oxaprozin),

(ii) Salicylic acid derivatives (e.g., aspirin, salsalate, sodium salicylate, choline magnesium trisalicylate, sulfasalazine, olsalazine, and diflunisal),

(iii) Anthranilic acids (e.g., mefenamic acid and meclofenamic acid),

(iv) Heteroaryl acids (e.g., tolmetin, diclofenac, and ketorolac),

(v) Enolic acids (e.g., piroxicam and meloxicam),

(vi) Indole and Indene acetic acids (e.g., indomethacin and sulindac),

(vii) Indole acteic acids (e.g., etodolac),

(viii) Para-Aminophenol derivatives (e.g., acetaminophen or Tylenol®)

(ix) Alkanes (e.g., Nabumetone),

(x) Diaryl-Substituted furanones (e.g., rofecoxib),

(xi) Diaryl-Substituted pyrazoles (e.g., celecoxib), and

(xii) Sulfonanilides (e.g., nimesulide).

The inventive cream also includes at least some of the natural ingredients listed below.

### Microencapsulated Water-Soluble USP Sulfur

Sulfur is a vital ingredient for dermatological products. Sulfur is an excellent natural preservative since it has anti-inflammatory, anti-bacterial and anti-fungal properties. Exemplary medical uses of sulfur preparations are as fungicides and parasiticides, and for the treatment of various cutaneous disorders such as psoriasis, seborrhoea, eczema-dermatitis, and lupus erythermatosus. Sulfur kills bacteria on and in the skin. Sulfur converts to pentatonic acids in order to exert germicidal activity. Sulfur also possesses a keratolytic property, which is the basic property needed to treat certain cutaneous disorders unassociated with infection. The inventive creams include between about 0% and about 8% by weight of microencapsulated USP sulfur.

### Microencapsulated Water-Soluble USP Zinc Oxide

Zinc oxide has been used in the treatment of literally hundreds of skin disorders and it has been at least partially successful in many of them. Zinc oxide has a mild astringent and antiseptic action. Zinc oxide is a Category I skin protector, and promotes healthy skin. Zinc oxide is used for treatment of skin diseases and infections such as eczema, impetigo, ringworm, varicose ulcers, pruritus, and psoriasis.

Zinc oxide regulates the activity of oil glands and is required for protein, DNA and RNA synthesis and collagen formation. Think oxide provides an excellent barrier to the sun and other irritants.

The inventors have produced microencapsulated USP zinc oxide in the form of the stable water-soluble emulsion/suspension for dermatological application. The common active ingredient used in derma cream compositions is zinc oxide. Zinc oxide in the form of fine particles (with a size of less than about 1 micron) is an astringent and antiseptic action principal remaining use is in the skin disorder treatment. The inventive creams include between about 0% and about 15% by weight of microencapsulated USP zinc oxide.

### Microencapsulated USP Vitamin C

Vitamin C (ascorbic acid) is reversibly oxidizable in the human body to a form known as dehydroascorbic acid. The physiological functions of vitamin C are related to this oxidation-reduction system. Vitamin C is involved in carbohydrate metabolism, reduction of glucose tolerance, and decrease of hepatic glycogen content and resistance to insulin. Vitamin C prevents the oxidation of epinephrine. A very important role of vitamin C is to decrease the rate of skin aging process and to increase the wound-healing process. Unique properties of vitamin C are that it: (i) stimulates: (1) blood circulation, (2) cell regeneration, (3) skin immune system, (4) fibroblasts and keratinocytes to produce collagen, and (5) dermal penetration of the active ingredients through the skin, (ii) is an anti-inflammatory agent, (iii) increases skin metabolism, (iv) activates dermal enzymes, and (v) promotes faster healing due to quicker absorption and prolonged duration. Vitamin C is an antioxidant that is required for at least 300 metabolic functions in the body, including tissue growth and repair, adrenal gland function, and healthy gums.

However, vitamin C is unstable and quickly oxidizes with time. In order to decrease the rate of oxidation and increase the biological activity of vitamin C, the inventors have produced microencapsulated USP Vitamin C in the form of a stable water-soluble emulsion for dermatological application. The inventive creams include between about 0% and about 4.5% by weight of microencapsulated vitamin C.

### Microencapsulated Water-Soluble Vitamin A

Vitamin A (Retinol A) has a number of important functions in the body. Vitamin A is essential for the integrity of epithelial cells and has a stabilizing effect on various membranes. Vitamin A also regulates membrane permeability and stimulates the synthesis of nuclear RNA, suggesting a role in genetic transcription and cell differentiation. Vitamin A is converted to a metabolite that is a true vitamin. Vitamin A is used for local treatments of infections, burns, and wounds.

Vitamin A is an anti-oxidant, which is a compound that may protect against disease by neutralizing unstable oxygen molecules, called free radicals, within the body. Vitamin A is involved in night vision, growth, cell differentiation and reproduction. Vitamin A also maintains the health of the skin (prevents acne and dermatitis) and surface tissues especially those with mucous linings. These linings are the body first defense against infection, which is why vitamin A helps fight colds and infections.

However, vitamin A is also unstable and quickly oxidizes with time. In order to decrease the rate of its oxidations and increase the biological activity of vitamin A, and the inventors have developed microencapsulated USP Vitamin A in the form of the stable water-soluble emulsion for dermatological application. The inventive creams include between about 0% and about 0.5% by weight of vitamin A.

### Natural Healing Oils

Natural healing oils included in the inventive creams may include blackcurrants seed oil, borage seed oil, dog rose hip oil, hazel nut oil, tamanu oil, sesame oil, chamomile oil, grape seed oil, lavender oil, clove oil, kiwi seed oil, tea tree oil, safflower oil, thymus oil. The inventive creams include between about 7.5% and about 30% by weight of healing oils.

### Herbal Extracts

Additionally, biological active herbal extracts included in the creams may include birch leave and bark extract, black nightshade extract, burdock extract, goldenseal extract, bilberry extract, calendula officinalis flower extract, carrot seed extract, comfrey leaf extract, grapefruit seed extract, olive leaves extract, St. John's wort extract, willow bark extract. The inventive creams include between about 0% and about 6% by weight of herbal extracts.

### Amino Acids

The inventive creams may also include essential and non-essential amino acids. The inventive creams may include between about 0% and about 6% by weight of microencapsulated amino acids.

### Other Ingredients

The inventive creams also include combinations of at least two of Decyl-glycoside (between about 0% and about 8% by weight), microencapsulated collagen peptides (between about 2% and about 6% by weight), menthol (between about 0% and about 1.5% by weight), camphor (between about 0% and about 2% by weight), salicylic acid (between about 0.3% and about 0.5% by weight), Aloe Barbadensis leaf extract (between about 0% and about 2% by weight), vitamin E (about 0.1% by weight), non-ionic surfactants and wetting agents (between about 0.01% and about 5% by weight), and water (between about 39.4% and about 79.4% by weight).

Exemplary compositions of the inventive creams are provided below. While the exemplary compositions listed below give percentages of ingredients by weight, it is contemplated that the weight percent of each ingredient may vary as much as ±5% of the total weight of the composition.

NATURAL BURN CREAM COMPOSITION

| Ingredients | Weight Percent (%) |
|---|---|
| microencapsulated Sulfur | 8 |
| microencapsulated Vitamin C | 2.5 |
| microencapsulated Vitamin A | 0.5 |
| microencapsulated collagen peptides | 2.0 |
| Menthol | 3.5 |
| Camphor | 3.0 |
| Healing Oil | 21 |
| Herbal Extract | 4.0 |
| microencapsulated Amino Acids | 3.0 |
| Salicylic Acid | 0.5 |
| vitamin E (da Tocopherol) | 0.1 |
| Water | 51.9% |

The inventors have found that the inventive burn cream generates a moist, anti-inflammation, anti-bacterial, and anti-microbial environment around a burn surface, thereby promoting fast recovery (in particular, suppurative burn) without infections. The inventive burn cream also decreases the risk of formation of colloid and hypertrophied scars and skin implantation.

NATURAL WOUND CREAM WITH VITAMIN C COMPOSITION

| Ingredients | Weight Percent (%) |
|---|---|
| microencapsulated Sulfur | 6 |
| microencapsulated Vitamin C | 3.0 |
| microencapsulated Vitamin A | 0.5 |
| microencapsulated collagen peptides | 2.0 |
| Menthol | 3.5 |
| Camphor | 3.0 |
| Healing Oil | 22 |
| Herbal Extract | 6.0 |
| microencapsulated Amino Acids | 3.0 |
| Salicylic Acid | 0.5 |
| Vitamin E (da Tocopherol) | 0.1 |
| Water | 50.4% |

The inventors have found that the inventive wound cream with microencapsulated vitamin C provides a moist, anti-inflammation, anti-bacterial, and anti-microbial environment around a wound surface, promoting a fast recovery (in particular suppurative wound) and reducing or even preventing skin infections. The combination of natural healing oils, natural wound cream with vitamin C, and ibuprofen cream dissolves solid pus and solid necrosis, promoting fast healing of deep wounds, in particular, diabetic and necrosis cases.

NATURAL ANTI-SCAR CREAM COMPOSITION

| Ingredients | Weight Percent (%) |
|---|---|
| microencapsulated Sulfur | 7 |
| microencapsulated Vitamin C | 4.5 |
| microencapsulated Vitamin A | 0.5 |
| microencapsulated collagen peptides | 6.0 |
| Camphor | 1.0 |
| Healing Oil | 20 |
| Herbal Extract | 4.0 |
| microencapsulated Amino Acids | 3.5 |
| Vitamin E (da Tocopherol) | 0.2 |
| Water | 53.3% |

The inventors have found that the inventive anti-scar cream provides soothing and softening effect on the skin, restoring skin to healthy state. The inventive cream diminishes and improves the overall appearance and texture of scars. The inventive cream may also help remove a scar without surgery.

NATURAL WOUND CREAM COMPOSITION

| Ingredients | Weight Percent (%) |
|---|---|
| microencapsulated Sulfur | 6 |
| microencapsulated Zinc Oxide | 3.5 |
| microencapsulated Vitamin A | 0.5 |
| microencapsulated collagen peptides | 2.0 |
| Menthol | 1.5 |
| Camphor | 2.0 |
| Healing Oil | 26 |
| microencapsulated Amino Acids | 3.0 |
| Herbal Extract | 6.0 |
| Salicylic Acid | 0.5 |
| Vitamin E (da Tocopherol) | 0.2 |
| Water | 48.8% |

The inventors have found that the inventive wound cream provides a moist, anti-inflammatory, anti-bacterial, and anti-microbial environment around wounds, cuts, and scraped skin surfaces to quickly heal (in particular suppurative burn and wound) and reduce or even prevent skin infections. Inventive wound cream provides nourishing and calming effects on damaged skin.

IBUPROFEN CREAM COMPOSITION

| Ingredients | Weight Percent (%) |
|---|---|
| microencapsulated Ibuprofen | 15 |
| Healing Oil | 25 |
| Herbal Extract | 1.0 |
| Vitamin E (da Tocopherol) | 0.1 |
| Water | 58.9% |

The inventors have found that the water-soluble ibuprofen cream provides a moist and anti-inflammatory environment around damaged skin surface, in particular suppurative damaged skin and also reduces or prevents skin infections. The inventors have further found that a combination of natural healing oils, natural wound cream with vitamin C, and ibuprofen cream dissolves solid pus and solid necrosis, and promotes quick recovery of deep wounds and burns, in particular, for diabetic and necrosis cases.

NATURAL ZINC OXIDE CREAM
FOR REGENERATING SKIN COMPOSITION

| Ingredients | Weight Percent (%) |
|---|---|
| Microencapsulated Sulfur | 5 |
| microencapsulated Zinc Oxide | 15.0 |
| microencapsulated collagen peptides | 5.0 |
| Healing Oil | 30 |
| Herbal Extract | 2.0 |
| Vitamin E (da Tocopherol) | 0.1 |
| Water | 42.9% |

The inventors have found that the inventive zinc oxide cream for regenerating skin regenerates damaged skin (i.e., wounds, 1st to 3rd degree burns, cuts).

NATURAL WOUND/BURN/INJURY/
SCAR CLEANER & MOISTURIZER

| Ingredients | Weight Percent (%) |
|---|---|
| Decyl-Glycoside | 8 |
| Healing Oil | 7.5 |
| Herbal Extract | 2.5 |
| Salicylic Acid | 0.3 |
| Aloe Barbadensis leaf extract | 2.0 |
| Vitamin E (da Tocopherol) | 0.1 |
| Water | 79.6% |

The inventors have found that the inventive wound/burn/injury/scar cleaner and moisturizer, in conjunction with dressing 100, occludes a burn/wound/injury/scar, controls infection level, and promotes healing by maintaining the burn/wound areas at a certain moisture level. The moist dressing system also uniformly distributes moisture, the inventive cleaner and moisturizer cream, and fluids through the burn/wound/injury/scar areas, and prevents or reduces the size and number of dry spots covering the burn/wound. The moist dressing 100 also promotes formation of granular tissue in the burn/wound/injury/scar, migration of epidermal cells, and help control infections resulting in faster closure of the burn/wound/injury/scar, less pain, and less scarring.

According to the present invention, the scar cleaner and moisturizer, used in conjunction with dressing 100 for treating burn/wound/injury/scar with the honeycomb pattern and drain channels, may provide the benefits of providing an optimum environment for burn/wound healing; providing pain relief; providing healing properties; does not adhere to the burn/wound/injury/scar; does not have or induce unpleasant odor; is sterilizable; has a high absorption capacity; allows monitoring of the burn/wound/injury/scar healing process; drains the blood and fluids from burn/wound/injury/scar; provides impermeability to exogenous bacteria, prevention of proliferation of burn/wound/injury/scar surface flora and reduction of burn/wound/injury/scar bacterial concentration; and does not produce a negative effect on burn/wound/injury/scar regeneration.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A bandage comprising:
a. a planar substrate having a first surface and a second surface opposed from the first surface;
b. a first matrix extending outwardly from the first surface, the first matrix having a first plurality of reservoirs having contiguous sides and arranged in a predefined pattern and having a first open portion disposed away from the substrate, the first open portion having a fluid disposed and retained therein; and
c. a second matrix extending outwardly from the first surface, the second matrix having a second plurality of reservoirs having contiguous sides and arranged in a predefined pattern and having a second open portion disposed away from the substrate, the second open portion having a fluid disposed and retained therein, the second matrix being separated from the first matrix by a drain channel,
wherein the fluid comprises a water-soluble water/oil/water emulsion.

2. The bandage according to claim 1, wherein the substrate further includes a longitudinal axis and wherein the drain channel extends obliquely to the longitudinal axis.

3. The bandage according to claim 2, wherein the drain channel extends at an angle between 30° and 60° relative to the longitudinal axis.

4. The bandage according to claim 3, wherein the drain channel extends at an angle of 45° relative to the longitudinal axis.

5. The bandage according claim 1, wherein the reservoir has a polygonal shape.

6. The bandage according to claim 5, wherein the shape is hexagonal.

7. The bandage according claim 6, wherein the hexagonal shaped forms a honeycomb pattern.

8. The bandage according claim 1, wherein the substrate comprises a translucent material.

9. The bandage according claim 8, wherein the substrate comprises a transparent material.

10. The bandage according claim 1, wherein the substrate comprises a polymer film.

11. The bandage according claim 1, wherein each of the first plurality of reservoirs and the second plurality of reservoirs has a height of between 1 mm and 5 mm.

12. The bandage according claim 1, further comprising a fluid disposed inside the first and second pluralities of reservoirs.

13. The bandage according to claim 12, wherein the fluid comprises a cream.

14. The bandage according to claim 12, wherein the cream comprises a water-soluble emulsion.

15. (canceled)

16. A method of treating a wound, the method comprising the steps of:
a. using the bandage of claim 1;
b. and
c. applying the bandage to a patient such that the fluid contacts the patient's skin and the drain channel extends in an oblique direction with respect to a longitudinal centerline of the patient.

17. (canceled)

18. (canceled)

19. The method according to claim 16, further comprising the step of allowing fluid to drain from the drain channel.

20. (canceled)

21. (canceled)

22. The bandage according to claim 16 wherein step c. comprises the cream keeping the patient's skin moist.
